(19) United States
(12) Patent Application Publication
(10) Pub. No.: US 2022/0408934 A1
(43) Pub. Date: Dec. 29, 2022

(54) Knee Pillow Assembly

(71) Applicant: Eric Mansour, Bronaugh, MO (US)

(72) Inventor: Eric Mansour, Bronaugh, MO (US)

(21) Appl. No.: 17/361,266

(22) Filed: Jun. 28, 2021

(51) Int. Cl. A47C 20/00

(52) U.S. Cl. CPC A47C 20/021

(57) ABSTRACT

A knee pillow assembly for relieving lower back pain while sleeping includes a sleeve that can be worn around a user's knee. The sleeve has a knee opening extending through the sleeve to accommodate the user's knee cap when the sleeve is worn around the user's knee. A pair of cushions is each coupled to the sleeve. Each of the cushions is offset with respect to the knee opening such that each of the cushions is positioned on an inner side of the user's leg when the sleeve is worn around the user's knee. Each of the cushions has a substantial thickness to space the user's legs apart from each other when the user sleeps on their side for relieving lower back pain.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to pillow devices and more particularly pertains to a new pillow device for relieving lower back pain while sleeping. The device includes a sleeve that is wearable around a user's knee and a pair of cushions that are attached to the sleeve. Each of the cushions is positioned between each of the user's knees to space the user's legs apart from each other when the user sleeps on their side. In this way the cushions relieve lower back pain that can result from sleeping on one's side.

### (2) Description of Related Art Including Information Disclosed Under 37 CFR 1.97 and 1.98

The prior art relates to pillow devices including a variety of knee pillow devices that are attachable to an inner side of a user's leg to space the user's legs apart from each other when the user is lying on their side. The prior art discloses a cushion that has wrapping elements for wrapping around a user's leg to space the user's legs apart from each other when the user is lying on their side. The prior art discloses a knee pad device for protecting a user's knee cap.

### BRIEF SUMMARY OF THE INVENTION

An embodiment of the disclosure meets the needs presented above by generally comprising a sleeve that can be worn around a user's knee. The sleeve has a knee opening extending through the sleeve to accommodate the user's knee cap when the sleeve is worn around the user's knee. A pair of cushions is each coupled to the sleeve. Each of the cushions is offset with respect to the knee opening such that each of the cushions is positioned on an inner side of the user's leg when the sleeve is worn around the user's knee. Each of the cushions has a substantial thickness to space the user's legs apart from each other when the user sleeps on their side for relieving lower back pain.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of a knee pillow assembly according to an embodiment of the disclosure.

FIG. 2 is a back perspective view of an embodiment of the disclosure.

FIG. 3 is a top view of an embodiment of the disclosure.

FIG. 4 is a cross sectional view taken along line 4-4 of FIG. 3 of an embodiment of the disclosure.

FIG. 5 is a perspective in-use view of an embodiment of the disclosure.

### DETAILED DESCRIPTION OF THE INVENTION

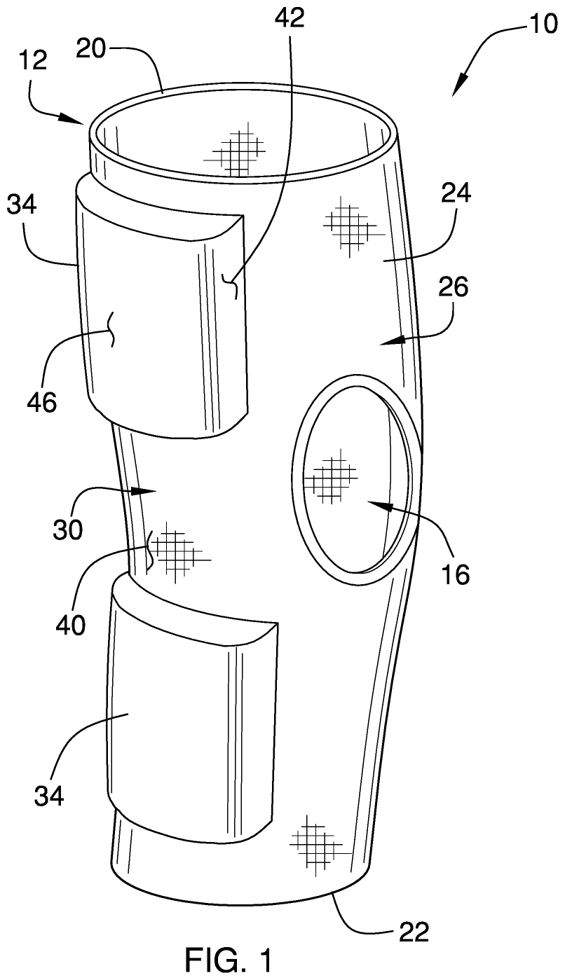

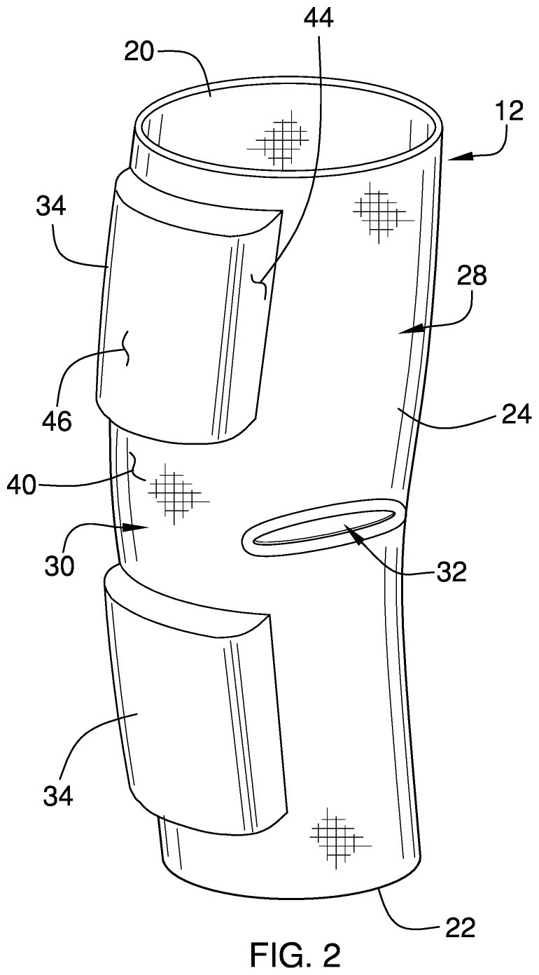

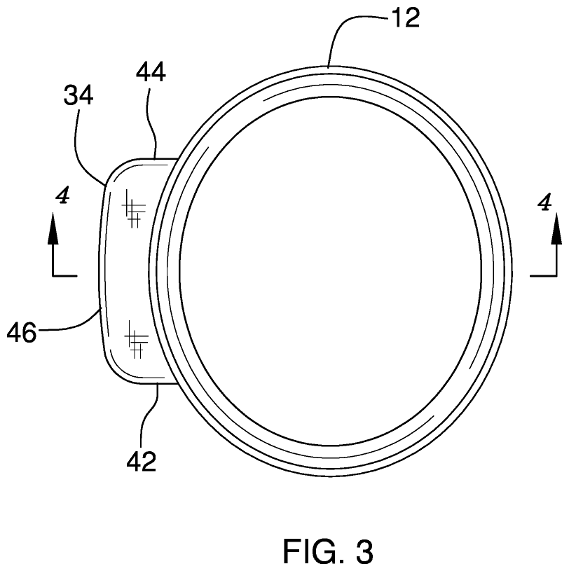

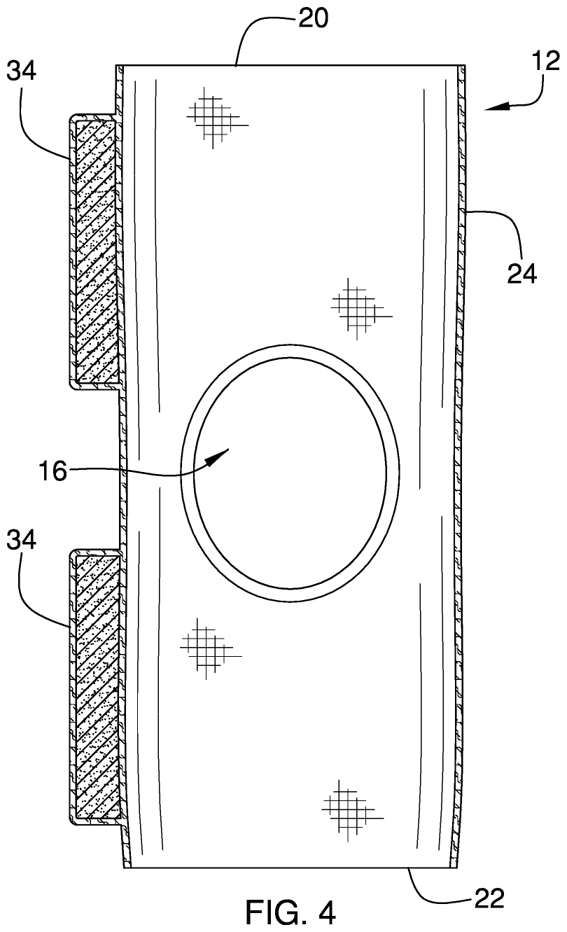

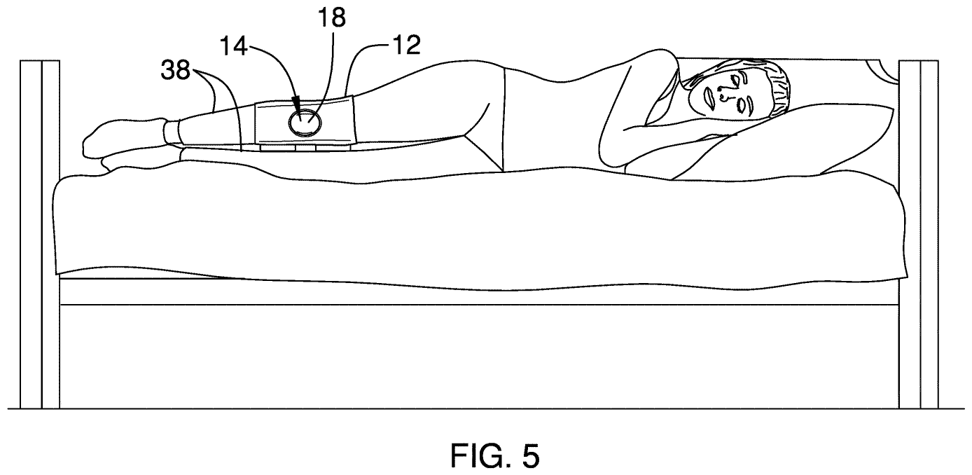

With reference now to the drawings, and in particular to FIGS. 1 through 5 thereof, a new pillow device embodying the principles and concepts of an embodiment of the disclosure and generally designated by the reference numeral 10 will be described.

As best illustrated in FIGS. 1 through 5, the knee pillow assembly 10 generally comprises a sleeve 12 that is comprised of a resiliently stretchable material such that the sleeve 12 can be worn around a user's knee 14. The resiliently stretchable material may comprise neoprene or other similar material that can be worn against a person's skin. The sleeve 12 has a first knee opening 16 extending through the sleeve 12 to accommodate the user's knee cap 18 when the sleeve 12 is worn around the user's knee 14. The sleeve 12 has a top end 20, a bottom end 22 and an outer wall 24 extending between the top end 20 and the bottom end 22, and the outer wall 24 has a front side 26, a back side 28 and a first lateral side 30. The first knee opening 16 extends through the outer wall 24 and the first knee opening 16 is positioned on the front side 26. Additionally, the first knee opening 16 is centrally positioned between the top end 20 and the bottom end 22. The outer wall 24 has a second knee opening 32 extending through the outer wall 24 and the second knee opening 32 is positioned on the back side 28 of the outer wall 24. In this way the second knee opening 32 is aligned with the back of the user's knee 14 to enhance comfort when the sleeve 12 is being worn.

A pair of cushions 34 is provided and each of the cushions 34 is coupled to the sleeve 12. Each of the cushions 34 is offset with respect to the first knee opening 16 such that each of the cushions 34 is positioned on an inner side 36 of the user's leg 38 when the sleeve 12 is worn around the user's knee 14. Each of the cushions 34 has a substantial thickness to space the user's legs 38 apart from each other when the user sleeps on their side for relieving lower back pain. Additionally, each of the cushions 34 is comprised of a resiliently compressible material to enhance comfort for the user.

Each of the cushions 34 is positioned on an outer surface 40 of the outer wall 24 of the sleeve 12 and each of the cushions 34 is positioned adjacent to a respective one of the top end 20 and the bottom end 22. Additionally, each of the cushions 34 is positioned on the first lateral side 30 of the outer wall 24 of the sleeve 12. In this way each of the cushions 34 can rest against the opposite leg 38 with respect to the leg 38 on which the sleeve 12 is positioned to space the user's legs 38 apart from each other. Each of the cushions 34 may have a thickness ranging between approximately 2.0 inches and 4.0 inches and a length ranging between approximately 6.0 inches and 10.0 inches. Additionally, the sleeve 12 may have a length ranging between approximately 18.0 inches and 22.0 inches and the sleeve 12 may be manufactured in a variety of diameters to accommodate different sizes of users.

Each of the cushions 34 has a front surface 42, a back surface 44 and an outer surface 46 extending between the front surface 42 and the back surface 44. An intersection between the front surface 42 and the outer surface 46 of the cushions 34 is rounded. An intersection between the back surface 44 and the outer surface 46 is rounded of the cushions 34 is rounded. Additionally, each of the front surface 42 and the back surface 44 slope toward each other between the first lateral side 30 of the sleeve 12 and the outer surface 40.

In use, the sleeve 12 is worn around the user's knee 14 when the user is going to sleep. The sleeve 12 is oriented such that each of the cushions 34 is positioned on the inner side 36 of the user's leg 38 on which the sleeve 12 is worn. In this way the cushions 34 rest against the user's opposite leg 38 when the user sleeps on their side. Thus, the cushions 34 space the user's legs 38 apart from each other to relieve lower back pain that can be caused when the user sleeps on their side. Additionally, the sleeve 12 and the cushions 34 alleviate the problem of using a pillow between the user's knees which constantly needs to be repositioned at the user moves during sleeping. In this way the user can enjoy continued relief from lower back pain when the user is sleeping.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A knee pillow assembly for holding a user's knees apart when the user is sleeping on their side to relieve lower back pain, said assembly comprising:
a sleeve being comprised of a resiliently stretchable material wherein said sleeve is configured to be worn around a user's knee, said sleeve having a knee opening extending through said sleeve, said knee opening being circular wherein said knee opening is configured to accommodate the user's knee cap when said sleeve is worn around the user's knee, said sleeve having a length between a top end and a bottom end; and
a pair of cushions, each of said cushions being coupled to said sleeve, each of said cushions being offset with respect to said knee opening wherein each of said cushions is configured to be positioned on an inner side of the user's leg when said sleeve is worn around the user's knee, each of said cushions having a substantial thickness wherein each of said cushions is configured to space the user's legs apart from each other when the user sleeps on their side for relieving lower back pain, each of said cushions being positioned adjacent to and inset from a respective one of said top end and said bottom end.

2. The assembly according to claim 1, wherein said sleeve has an outer wall extending between said top end and said bottom end, said knee opening extending through said outer wall, said outer wall having a front side and a first lateral side, said knee opening being positioned on said front side, said knee opening being centrally positioned between said top end and said bottom end.

3. The assembly according to claim 2, wherein each of said cushions is positioned on an outer surface of said outer wall of said sleeve, each of said cushions being positioned on said first lateral side of said outer wall of said sleeve wherein each of said cushions is configured to rest against the opposite leg with respect to the leg on which said sleeve is positioned to space the user's legs apart from each other.

4. The assembly according to claim 1, wherein each of said cushions has a front surface, a back surface and an outer surface extending between said front surface and said back surface, an intersection between said front surface and said outer surface being rounded, an intersection between said back surface and said outer surface being rounded, each of said front surface and said back surface sloping toward each other between a first lateral side of said sleeve and said outer surface.

5. A knee pillow assembly for holding a user's knees apart when the user is sleeping on their side to relieve lower back pain, said assembly comprising:
a sleeve being comprised of a resiliently stretchable material wherein said sleeve is configured to be worn around a user's knee, said sleeve having a knee opening extending through said sleeve, said knee opening being circular wherein said knee opening is configured to accommodate the user's knee cap when said sleeve is worn around the user's knee, said sleeve having a top end, a bottom end and an outer wall extending between said top end and said bottom end, said knee opening extending through said outer wall, said outer wall having a front side and a first lateral side, said knee opening being positioned on said front side, said knee opening being centrally positioned between said top end and said bottom end; and
a pair of cushions, each of said cushions being coupled to said sleeve, each of said cushions being offset with respect to said knee opening wherein each of said cushions is configured to be positioned on an inner side of the user's leg when said sleeve is worn around the user's knee, each of said cushions having a substantial thickness wherein each of said cushions is configured to space the user's legs apart from each other when the user sleeps on their side for relieving lower back pain, each of said cushions being positioned on an outer surface of said outer wall of said sleeve, each of said cushions being positioned adjacent to and inset from a respective one of said top end and said bottom end, each of said cushions being positioned on said first lateral side of said outer wall of said sleeve wherein each of said cushions is configured to rest against the opposite leg with respect to the leg on which said sleeve is positioned to space the user's legs apart from each other, each of said cushions having a front surface, a back surface and an outer surface extending between said front surface and said back surface, an intersection between said front surface and said outer surface being rounded, an intersection between said back surface and said outer surface being rounded, each of said front surface and said back surface sloping toward each other between said first lateral side of said sleeve and said outer surface.
